(19) United States
(12) Patent Application Publication
(10) Pub. No.: US 2013/0149395 A1
(43) Pub. Date: Jun. 13, 2013

(54) Pharmaceutical Product, Method of Production and Method of Application of the Pharmaceutical Product

(71) Applicants: Shalva Iosifovitch Mardi, Binningen-BL (CH); Lev Alexandrovich Ustynyuk, Moscow (RU)

(72) Inventors: Shalva Iosifovitch Mardi, Binningen-BL (CH); Lev Alexandrovich Ustynyuk, Moscow (RU)

(21) Appl. No.: 13/805,472

(22) PCT Filed: Apr. 22, 2011
(86) PCT No.: PCT/RU2011/000260

§ 371 Date: Feb. 21, 2013

(30) Foreign Application Priority Data
Dec. 9, 2010 (RU) 2010150315

(51) Int. Cl. A61K 47/20

(52) U.S. Cl. CPC A61K 47/20
USPC 424/702

(57) ABSTRACT

The claimed pharmaceutical product is obtained by interaction between a selenium-containing compound with alpha, alpha-dichlorocarboxylic acid, stabilized by nutric acid with the amount of nitric acid not exceeding 5 pts. wt., preferably 1-3 pts. As a selenium-containing compound the solution of the selenious acid in the amount of no more than 20 pts. wt., preferably 0.5-10.0 pts. wt. is used during the interaction. The product can also contain additional 5-20% of dimethylsulfoxide. The production process of the pharmaceutical product is carried out by interaction between a selenium-containing compound with alpha, alpha-dichlorocarboxylic acid, stabilized by nutric acid with the amount of nitric acid not exceeding 5 pts. wt., preferably 1-3 pts., and as a selenium-containing compound the solution of the selenious acid in the amount of no more than 20 pts. wt., preferably 0.5-10.0 pts. wt. is used during the interaction, which is carried out at the temperature not exceeding 70° C., preferably at 20-30° C. The pharmaceutical product used for the treatment of benign, viral, pre-malignant and malignant non-metastatic skin lesions, dysplastic lesions of visible mucous coats and other skin lesions, should be applied to the lesion focus on days 1, 2-3, 7-9 and 22-24 of a treatment.

One of the prevailing methods of the tumor skin lesions treatment involves the usage of various preparations based on mineral and organic acids, which are well-known for the amystic (necrotising) effect they produce on skin and mucus membranes. However, many of the preparations suggested are unstable and can change its composition in course of time, hereupon no required curative effect is achieved: neoplasm eradication isn't complete, the recurrence rate is high. In addition, a number of side and negative effects (in particular, the lesions of intact skin areas, covering the topically-treated neoplasma focus) and long rehabilitation period are degrading the life quality of patients after the conducted procedures.

The present invention is intended for the treatment of benign, viral, premalignant and malignant non-metastasizing skin lesions, of dysplastic lesions of visible mucous membranes, in rhytidectomy, for the treatment of senile lentigos, fungous and other skin diseases.

The statistic analysis shows, that over the last decade the rate of nonmalignant and malignant neoplasms related diseases among the Russian Federation population has been constantly growing (A. A. Kubanova, A. A. Martynov. The role of malignant skin neoplasms in the structure of cancer morbidity in the Russian Federation. Dermatology and Veneorology Bulletin, 2007,vol. 6, pp. 19-24). Therefore invention of new and more effective preparations for the treatment of tumors and other skin diseases is a relevant objective nowadays.

U.S. Pat. No. 7,128,903 suggests a preparation, consisting of trichloracetic, hydrochloric and formic acids (U.S. Pat. No. 7,128,903, 2001. Burstein P. Pharmaceutical preparations useful for treating tumors and lesions of the skin and the mucous membranes and methods and kits using same), whilst U.S. Pat. No. 5,407,958 and U.S. Pat. No. 4,380,549 suggest preparations based on alpha-hydroxycarboxylic acid (U.S. Pat. No. 5,407,958, 1993. Heath J. L, Sanders C. R, Murphy J. H., Atkins R. Therapeutic skin composition; U.S. Pat. No. 4,380,549, 1975. Van Scott E. J., Yu Ruey J., Topical treatment of dry skin). It is proved, however, that these acids reduce the skin pH up to the values lower than 3.0, whereupon the skin integument condition is declining. The substitution of free acids for their salts with alkylamines or alkylaminespirits causes a weakening or a complete loss of the therapeutic effect.

In order to increase the preparation activity, either salicylic acid or acids of zinc, cuprum, cadmium and other metals are usually added to the mixture of carboxylic acids (U.S. Pat. No. 5,573,786, 1993. Grabo M., Staehli Ch., Meyer R., Glauser R. E., Weiner M. Preparation for skin and mucous membrane; U.S. Pat. No. 7,258,875, 2003. Chiou Win L. Compositions and methods for topical treatment of skin infection) or there can be used compounds, containing nitric (and nitrous) acid (U.S. Pat. No. 4,595,591, 1979. Mardi Sh., Lichti H. F., Baumgartner G., Garteiz D., Judd C. I., Weiner M. Use of dilute nitric acid solutions for treating certain skin lesions) or aliphatic haloid carboxylic acids; Russian Federation Patent 2261243, 2004. Mardi Sh., Tsib A. F., Krikunova L. I. Zinc and haloid carboxylic acids of aliphatic series salts for the treatment of skin lesion and visible mucus membranes; Russian Federation Patent 2375054, 2007. Mardi Sh., Tsib A. F., Preparation for the skin lesions treatment, its production (various ways) and application).

Also offered for the treatment of a number of skin diseases are complex selenium-containing compounds with haloacetic acids (EP 1293498, 2001. Mardi Sh., Mardi R., Mardi G., Mardi L. Selenium complex with haloethanoic acid or its anhydride, and use thereof in the topical treatment of neoplasms), whereas for the prophylaxis and treatment of viral diseases aqueous solutions Na₂SeO₃, stabilized by the addition of acetic, citric, malic and other acids are suggested (the Russian Federation patent 2277915, 2000. Cessler P., Fuks N., Kuklinski B., Shipher R. The method of increasing the antioxidative potential of selenium-containing aqueous solutions).

The closest analogue of the present invention in terms of the technical characteristics is the Russian Federation patent 2366648 (Russian Federation patent 2366648, 2007. Mardi Sh. The product of interaction between selenious dioxide and aliphatic haloid carboxylic acids, the method of product preparation and the method of treatment of benign, viral, pre-malignant and malignant non-metastasizing skin lesions, of the dysplastic lesions of visible mucous coats and other skin lesions). According to this patent, the preparation of complex compounds of general formula H₂SeO₃ x RCXY(CH₂)ₘCOOH is achieved by heating selenious dioxide with aliphatic haloid carboxylic acids, followed by filtering and precipitation.

The disadvantages of this method are the instability of solutions of the semi-finished product (substance) and the dosage band, low and uncontrollable concentration of selenium and the impossibility of its regulation. The compositions recommended for the practical application, were resolving while in storage, and the process was accompanied by the selenium precipitation, which is unacceptable. The precipitation especially accelerated when diluting semi-finished product (substance) with water.

The interaction between the pharmaceutical product and water is a pattern of behavior of the preparation in treatment of a skin lesion area. It is widely thought, that the destruction of the neoplasm when affected by acid-based compositions starts from the deaquation of epithelial cells. The precipitation of selenium at this stage practically excludes the latter from the treatment process, preventing its transfer into the neoplasm. Moreover, there is information that in case of a direct contact between selenium and skin, the breaking-out of dermatitis in a form of rash, skin burns with a considerable induration of skin tissue, violent pains and numbness are possible. (A. Avtsyn, A. Zhavoronkova, M. Rish, L. Strochkova. Human microelementoses (ethiology, classification, organopathology). Academy of Medical Sciences USSR, M., Medicine, 1991, 496 p.; I. Ognerubova, I. Poddubnaya. The application of selenium in oncology. Clinical oncology, 11, vol. 2, 2009). Thus, not only does the precipitation of selenium reduce the therapeutic effect of the medication, but it also contributes to the appearance of the unwanted sequela of its application.

The principal object of this invention is the further development of medications for the treatment of benign, viral, premalignant and malignant non-metastatic skin lesions, dysplastic lesions of visible mucus membranes, fungous and other skin diseases by formation of a therapeutic agent and pharmaceutical product, which is distinguished by a high stability during the entire life cycle, pathotropism to tumors (without the destruction of the adjacent skin tissues) and a high penetration capability.

Precipitation when storing pharmaceutical substances is a typical example of chemical incompatibility, and in order to overcome this incompatibility, either pharmaceutical aids are added to the formula or special packaging is used.

To specify causes of instability, claimed in the prototype of medicinal agent composition, it was necessary, first of all, to determine with which of the compounds (impurities) of the intact haloid carboxylic acids selenious acid and its anhydride can react.

As it was shown the selenious dioxide is widely used in special organic synthesis for selective oxidation of activated methyl or methylene groups into corresponding aldehydes or ketones (e.g.: CH₃CHO into glyoxal, cyclohexanone—into cyclohexandion-1.2 and etc.) (Preparative Organic Chemistry, translated from Polish by V. V. Shpanova and V. S. Volodina, published by Himicheskaya Literatura, Moscow, 1959, p. 667). Therefore, aliphatic carboxylic acids containing such groups (if they are found in raw material), will also be subjected to oxidation.

The impurity identification of 2,2-dichloropropionic acid, taken as an example, has been conducted using the method of NMR spectroscopy on the ¹H and ¹³C nuclei, one of the most informative methods, which allows to determine the composition of each impurity even if the numerous related compounds are found in the sample.

It is proved that 2,2-dichloropropionic acid is daylight-sensitive and can transform into pyroracemic acid or eliminate HCl, followed by the production of alpha chloroacrylic acid during prolonged storage:

CH₃COCOOH←CH₃CCl₂COOH→CH₂═CClCOOH

Warranty storage life of the reagents grade product does not exceed 2 years.

The incoming inspection of starting materials has shown that the mass content of 2,2-dichloropropionic acid in commercial products does not practically exceed 80%. The main impurities are related acids: 2-chloropropionic acid and dichloroacetic acid (up to 5%), acetic acid, chlorine—and trichloroacetic acid, propionic acid, tri and tetra-chloropropionic acids, cis-chloroacrylic and pyruvic acids (the content of each varies from 0.05 to 1-2%), and a number of minor impurities at a rate of about 0.01%, which were not identified.

According to the U.S. Pat. No. 3,772,157, 1971. Horsley L., Purification of 2-chloroalkanoic acids by azeotropic distillation) and 5,215,671 (U.S. Pat. No. 5,215,671, 1990. Ono Yuzo, Kajikuri Thutomu., Kidano Shoji, Senoo Kenji. Purification method of 2-chloropropionic acid), the most effective way of separating the impurities of 2-chloropropionic acid from 2,2-dichloropropionic acid and vice versa is azeotropic distillation in a vacuum of not less than 10 mm of mercury at column efficiency up to 20 TT (theoretical plates) with reflux-to-product ratio 0.5-1.5. In order to obtain small amounts of 2,2-dichloropropionic acid of purity grade over 97%, the method of its purification using cyclohexylammonium H. A. J. Holterman and J. B. F. N. Engberts. J. Org. Chem. 1983, 48, 4030-4035) and plumbic salts was also recommended. However, according to our data the usage of 2,2-dichloropropionic acid, purified by the afore-mentioned methods, does not guarantee the stabilization of the preparation.

A number of special tests be accomplished, it was found out that after several months of room temperature storage there is almost no signals of acetic and propionic acids in the spectrum of solution of the specimen based on 2,2-dichloropropionic acid, whereas signals of glyoxalic and oxalic acids do show up. The simultaneous precipitation of selenium is observed. After the successive filtration of selenium precipitation it precipitates again after a while this time from the clarified solution, which indicates the incompleteness of the redox process due to its low speed.

NMR spectroscopy method on ⁷⁷Se nuclear of solution analysis proved the formation of a complex of selenious acid with 2,2-dichloropropionic acid.

It was found that if the content of water in the preparation is low (less than 1%), then, out of 4 signals of low intensity with chemical shifts of 1116.4, 1040.8, 662.3 and 439.6 ppm, only the first two can be reliably attributed to derivatives containing the arrangement Se═O and evidently representing mixed anhydrides of selenious and 2,2-dichloropropionic acid of composition CH₃CCl₂COOSeO(OH) and (CH₃CCl₂COO)₂Se═O, and the last two signals apparently belong to the complexes of these mixed anhydrides with 2,2-dichloropropionic acid.

Addition of 10% amount of water to the sample leads to complete disappearance of all the 4 signals and to appearance of one intense signal at 1236 ppm. Compare: aqueous solution of selenious acid contains only one signal at 1279 ppm, and solution of selenious acid in 2,2-dichloropropionic acid contains only one signal at 1236 ppm. The shift of ⁷⁷Se signal of 43 ppm towards the stronger field is indicative of interaction between those acids, which, apparently, is being accompanied by the formation of complexes like H₂SeO₃.x CH₃CCl₂COOH (coefficient x is not identified experimentally),) for no other changes have been detected in the composition CH₃CCl₂COOH.

Therefore, the problem of Se-containing preparations stability is limited to the elimination of chemical incompatibility between selenious acid (or its equivalents) and those components of the medicinal agent that act as deoxidants.

The problem in question has been solved by the present invention.

In production of the medicinal agent obtained by means of interaction between selenium-containing compound and alpha, alpha-dichlorocarboxylic acid, according to the invention, the selenious acid solution and alpha, alpha-dichloropropionic acid (stabilized by nitric acid with the amount of nitric acid no more than 5 pts. wt.) are used as the selenium-containing compound; the content of selenium in the end product may range from 0.5 to 10 pts. wt. The mass content of nitric acid advantageously ranges from 1 to 3 pts. wt. The mass content of selenium in the medication advantageously ranges from 0.5 to 5 pts. wt.

The pharmaceutical product may additionally contain dimethylsulfoxide in the quantity of 5-20%.

The pharmaceutical product is obtained by interaction of selenium-containing compound with an alpha, alpha-dichlorocarboxylic acid, according to the invention the interaction is conducted at a temperature no higher than 70° C., preferably at 20-30° C.

In the process of pharmaceutical preparation dichloroacetic, 2,2-dichlorobutyric acids and other members of the homologous series subjected to a similar stabilization can be used as an alternative to alpha, alpha-dichlorocarboxylic acid.

The distinctive features of the invention are:

processing of 2,2-dichloropropionic acid by nitric acid at isothermal conditions, during which attendant impurities of acetic, propionic, chloroacrylic, glyoxalic and pyruvic acids oxidize with the release of the gaseous products (CO, CO2, NO [symbol] NO2) and are removed from the reaction mixture.

addition of the selenious acid aqueous solution to 2,2-dichloropropionic acid thus avoiding the formation of unstable to the moisture mixed anhydrides and resulting in a production of a preparation where selenium is not precipitated during the warranty storage period of the product.

presence of a more powerful oxidant in the system that leads to almost complete suppression of redox potential of selenious acid and ensures its high stability.

The achieved result is confirmed by NMR spectra on the ¹H and ¹³C nuclei, according to which the resulting preparation has virtually no signals corresponding to acetic, propionic, chloroacrylic, glyoxalic and pyruvic acids. The relative content of attendant chlorocarboxylic acids remains at the initial level, indicating that they do not react with either nitric or selenious acid.

The advantages of the method are:

sufficiently simple technique was used to remove all the admixture oxidizable by the selenious acid from the reacting system,

when diluting strong solution of the semi-finished product (substance) the dosage form preserves stability, which hadn't been noticed before,

when using the selenious acid the content of selenium in the solution can be raised up to any reasonable values,

selenium preservation in the preparation can also be seen when applying the preparation to the skin lesion area, which provides the necessary therapeutic effect.

The essential feature of the invention lies in the increase of the efficacy of pharmaceutical product achieved by adding dimethyl sulfoxide to its compound in the quantity of 5-20%. It is mixed with the preparation in every respect and enables its skin, mucous and biological membrane penetration, which in some cases allows to reduce the period of treatment. Dimethylsulfoxide is widely used as a processing aid for pharmaceutical preparations; it is less toxic than saline solution, and that's why its admixtures in the preparation are not limited.

Being used as a pharmaceutical product for treating benign, viral, pre-malignant, malignant non-metastasizing skin lesions, the dysplastic lesions of the visible mucous coats and other skin diseases, it is applied to the lesion focus on the 1, 2-3, 7-9 and the 22-24 days of treatment.

The present invention can be illustrated by the following examples:

### EXAMPLE 1

### Production of the Prototype

The prototype is produced by adding 5 ml of distilled water and 0.66 g (0.006 g-mole) of selenium dioxide to 45 ml of 2,2-dichloropropionic acid (62.5 g, 0.437 g-mole) then heating the substance at a temperature of 105° C. while stirring for 60 min., cooling it to room temperature, filtering it until yellow transparent solution is obtained. Quality parameters are: d₄²⁰ 1.362, 2,2-dichloropropionic acid content—91.9%, water—7.8%, selenium—0.1%.

When storing semi-finished product (substance) at 20-25° C. for 3 months selenium precipitates from the preparation. Quality parameters are: d₄²⁰ 1.361, 2,2-dichloropropionic acid content—92.0%, water—7.9%, selenium—0.03%.

When diluting the semi-finished product with distilled water to a concentration of 70%, selenium comes down as a red precipitate after 5 hours, turning gray in a few days. Quality parameters are: d₄²⁰ 1.278, 2,2-dichloropropionic acid content—70.5%, water—28.8%, selenium—0.03%.

### EXAMPLE 2

### Production of the Semi-Finished Product and the Pharmaceutical Product According to the Invention

The production is carried out by adding in driblets while stirring 1.7 ml (2.3 g, 1 mass %) of 60% nitric acid to 100 ml of the 2,2-dichloropropionic acid with the density of d₄²⁰ 1.393 and purity grade no less than 98.5%, then the product is held under isothermal conditions until complete cessation of the gas release occurs, subsequently followed by the addition of H₂SeO₃ solution, obtained by dissolving 1.435 g of SeO₂ in 10 ml of water. The produced solution is stirred at the temperature of 50-60° for 12 hours and in result 151.5 g of transparent yellow solution of semi-finished product is obtained. Quality parameters are: d₄²⁰ 1.386, content of 2,2-dichloropropionic acid—91.7%, of water—7.8%, of selenium—0.68%, nitrate ions—0.04%.

The semi-finished product is then diluted with distilled water to a concentration of 70% and thus a pharmaceutical product in the form of a colorless solution is obtained. Quality parameters are: d₄²⁰ 1.279, content of 2,2-dichloropropionic acid—70.5%, of water—29.3%, of selenium—0.49%, nitrate ions—0.03%.

Other examples from this series are shown in the table 1.

TABLE 1
Synthesis of the semi-finished product and pharmaceutical product,
stabilized by the nutric acid additions

| | | Synthesis | quality data | | | |
|---|---|---|---|---|---|---|
| | Content | temperature, | | 2,2-DcpA , | | |
| [symbol] | HNO₃, % | ° C. | d₄²⁰ | % | Se,% | NO₂⁻,% |

Semi-finished Product Synthesis

| | | | | | | |
|---|---|---|---|---|---|---|
| 2-1 | 1 | 20 | 1.386 | 92.7 | 0.68 | 0.04 |
| 2-2 | 1 | 50 | 1.385 | 92.5 | 0.68 | 0.04 |
| 2-3 | 1 | 70 | 1.387 | 92.6 | 0.67 | 0.04 |
| 2-4 | 3 | 20 | 1.387 | 93.4 | 0.69 | 0.08 |
| 2-5 | 3 | 50 | 1.388 | 93.2 | 0.70 | 0.09 |
| 2-6 | 3 | 70 | 1.388 | 93.2 | 0.67 | 0.09 |
| 2-7 | 5 | 20 | 1.386 | 95.5 | 0.69 | 0.13 |
| 2-8 | 5 | 50 | 1.386 | 95.3 | 0.65 | 0.11 |

Pharmaceutical Product Synthesis

| | | | | | | |
|---|---|---|---|---|---|---|
| 2-9 | From the | 20 | 1.279 | 70.5 | 0.48 | 0.03 |
| | semi- | | | | | |
| | finished | | | | | |
| | product 2-1 | | | | | |
| 2-10 | From the | 20 | 1.279 | 70.0 | 0.47 | 0.04 |
| | semi- | | | | | |
| | finished | | | | | |
| | product 2-6 | | | | | |
| 2-11 | From the | 20 | 1.279 | 70.8 | 0.46 | 0.04 |
| | semi- | | | | | |
| | finished | | | | | |
| | product 2- 8 | | | | | |

### EXAMPLE 3

### The Preparation of the Semi-Finished Product with High Concentration of Selenium

The synthesis of the semi-finished product is conducted in the same way as shown in the example 2 with the only difference in the quantity of the selenious acid. The examples of this type are shown in the table 2.

TABLE 2
The synthesis of the semi-finished product with the high concentration
of selenium.

| | | quality data of the semi-finished product | | | | |
|---|---|---|---|---|---|---|
| [symbol] | H₂SeO₃, % | d₄²⁰ | 2.2-DcpA, % | Water, % | Se,% | NO₃⁻,% |
| 3-1 | 0.5 | 1.390 | 92.5 | 7.5 | 0.32 | 0.04 |
| 3-2 | 1 | 1.391 | 92.7 | 7.5 | 0.61 | 0.04 |
| 3-3 | 2 | 1.386 | 91.3 | 8.0 | 1.23 | 0.04 |
| 3-4 | 5 | 1.387 | 91.1 | 8.5 | 3.05 | 0.05 |
| 3-5 | 10 | 1.390 | 92.1 | 7.6 | 6.15 | 0.04 |
| 3-6 | 16 | 1.394 | 92.6 | 7.3 | 9.81 | 0.03 |
| 3-7 | 20 | 1.393 | 91.8 | 7.9 | 12.26 | 0.05 |

### EXAMPLE 4

### The Preparation of the Medical Agent with High Permeability

100 ml of the semi-finished product obtained as shown in ex. 2 is mixed with 10 ml of dimethylsulfoxide and diluted by distilled water to a concentration of 70%.

Quality parameters are: d₄²⁰ 1.278, the quantum of 2,2-dichloropropionic acid—70.3%, of water—29.5%, of selenium—0.49%.

The other examples of this kind are shown in the table 3.

TABLE 3
The synthesis of the medical agent with high permeability

| | | | H₂SeO₃ in the | quality data | | | |
|---|---|---|---|---|---|---|---|
| | | HNO₃ in the | semi-finished | | 2,2- | | |
| | (CH₃)₂SO, | semi-finished | product, | | DcpA, | | NO₃⁻, |
| [symbol] | % | product, % | % | d₄²⁰ | % | Se,% | % |
| 4-1 | — | 1 | 0.5 | 1.279 | 70.2 | 0.21 | 0.05 |
| 4-2 | 20 | 3 | 1 | 1.278 | 70.0 | 0.48 | 0.04 |
| 4-3 | 20 | 5 | 2 | 1.278 | 70.3 | 0.85 | 0.04 |
| 4-4 | 20 | 1 | 5 | 1.280 | 70.1 | 2.15 | 0.04 |
| 4-5 | 10 | 3 | 10 | 1.283 | 70.1 | 4.31 | 0.05 |
| 4-6 | 5 | 3 | 20 | 1.288 | 70.2 | 8.59 | 0.03 |

The analysis of the solution stability was held with the help of the method of accelerated aging of specimen kept in the thermostat at the temperature of 50° C. and in real storage conditions at the temperature of 20-25° C. The results are shown in table 4.

TABLE 4
The solution stability of the semi-finished product and pharmaceutical product under extended
storage conditions

| | | | | | | quality data | | | | |
|---|---|---|---|---|---|---|---|---|---|---|
| | | | | | Physical | | | 2,2- | | |
| | Tempera- | | | Monitored | Configura- | | | DcpA , | | NO₃⁻, |
| [symbol] | ture ° C. | Duration | | solution | tion | d₄²⁰ | H₂O | % | Se,% | % |
| 1 | 20-25 | 3 | mo | Semi-finished | settling | 1.361 | 7.9 | 92.0 | 0.03 | — |
| | | | | product | precipitated | | | | | |
| | | | | prototype | | | | | | |
| 2 | 20-25 | 5 | hrs | pharmaceutical | settling | 1.278 | 28.8 | 70.5 | 0.03 | — |
| | | | | product | precipitated | | | | | |
| | | | | prototype | | | | | | |
| 3 | 20-25 | 2 | yr | Semi-finished | no change. | 1.386 | 7.5 | 92.7 | 0.68 | 0.03 |
| | | | | product | no settling | | | | | |
| | | | | 2-1. 1% HNO₃ | | | | | | |
| 4 | 50 | 9 | mo | Semi-finished | no change, | 1.387 | 6.7 | 93.2 | 0.69 | 0.08 |
| | | | | product | no settling | | | | | |
| | | | | 2-6. 3% HNO₃ | | | | | | |
| 5 | 50 | 9 | mo | Semi-finished | no change. | 1.386 | 4.6 | 95.3 | 0.65 | 0.11 |
| | | | | product | no settling | | | | | |
| | | | | 2-8, 5% HNO₃ | | | | | | |
| 6 | 20-25 | 2 | yr | pharmaceutical | no change. | 1.279 | 29.3 | 70.5 | 0.48 | 0.03 |
| | | | | product 2-9 | no settling | | | | | |
| 7 | 20-25 | 2 | mo | pharmaceutical | no change, | 1.279 | 29.7 | 70.0 | 0.47 | 0.04 |
| | | | | product | no settling | | | | | |
| | | | | 2-10 | | | | | | |
| 8 | 50 | 9 | mo | pharmaceutical | no change. | 1.279 | 29.2 | 70.8 | 0.46 | 0.04 |
| | | | | product | no settling | | | | | |
| | | | | 2-11 | | | | | | |
| 9 | 20-25 | 2 | yr | Semi-finished | no change. | 1.393 | 3.6 | 93.5 | 3.05 | 0.03 |
| | | | | product | no settling | | | | | |
| | | | | 3-4. 5% H₂SeO₃ | | | | | | |
| 10 | 50 | 9 | mo | Semi-finished | no change, | 1.393 | 7.5 | 92.1 | 7.6 | 0.04 |
| | | | | product | no settling | | | | | |
| | | | | 3-5. | | | | | | |
| | | | | 10% H₂SeO₃ | | | | | | |
| 11 | 20-25 | 2 | yr | Semi-finished | no change, | 1.395 | 8.0 | 91.8 | 7.9 | 0.05 |
| | | | | product | no settling | | | | | |
| | | | | 3-6, | | | | | | |
| | | | | 20% H₂SeO₃ | | | | | | |
| 12 | 50 | 9 | mo | pharmaceutical | no change, | 1.278 | 29.5 | 70.2 | 0.65 | 0.04 |
| | | | | product 4-1 | no settling | | | | | |
| 13 | 20-25 | 2 | yr | pharmaceutical | no change, | 1.278 | 29.4 | 70.3 | 1.32 | 0.04 |
| | | | | product | no settling | | | | | |
| 14 | 20-25 | 2 | yr | pharmaceutical | no change, | 1.280 | 29.4 | 70.5 | 1.33 | 0.04 |
| | | | | product 4-3 | no settling | | | | | |
| 15 | 20-25 | 2 | yr | pharmaceutical | no change, | 1.283 | 29.5 | 70.1 | 4.31 | 0.05 |

| | |
|---|---|
| product 4-5 | no settling |

Method of the Pharmaceutical Product Application is as Follows:

As the medicinal agent, the standard selenious acid solution (0.45% of selenium) is used in 70% 2,2-dichloropropionic acid or, alternatively, a preparation with an additive of 10-30% dimethylsulfoxide is used.

The tests of the preparation were carried out on different groups of patients: the first group involved patients diagnosed with basal cell carcinoma (BCC), the second one involved patients with benign skin lesions of viral origin, benign neoplasms and vascular benign diseases of skin.

Basal cell carcinoma (basal cell epithelioma, basalioma) is a malignant epithelial skin tumor with local invasive destructive growth and extremely rare metastasis.

The treatment was outpatient without anesthesia application. The preparation was applied to lesion foci (after the preliminary treatment with 70% ethanol) with a plastic spatula or a glass capillary, covering additional 1-2 mm of healthy skin, until the appearance of white-grey coloring.

The dosage of the preparation depends on the stage, clinical form of the disease and the density of the tumor, though usually the single curative dose did not exceed 0.2 ml. The treatment period is 1 course (3 weeks). The standard therapeutic course of the 1st stage basal cell carcinoma included 3 single applications of the pharmaceutical product on the tumor on the 1st day, on the 2-3rd and 8-9th days. Next day after the application the pathological focus partially mummifies, reduces in size drastically and turns dark brown. The healing process goes without complications or without leaving any significant scars, cicatrices or deformations of adjacent tissues, or any damages to internal functions. At the end of the therapeutic course a thorough observation of tissues takes place after complete mummified scab has been rejected, and if there is a suspicion of the incompleteness of the treatment an additional 4^{th} application may be prescribed by the doctor.

25 patients (5 men and 20 women), aged 29 to 79, with the “primary basal cell carcinoma (BCC), stage T1N0M0” diagnosis formed a test group. The diagnosis was confirmed by the results of the cytological screening. In 8 cases the superficial form was diagnosed, in 12—nodular form and in the other 5—the ulcerous form of BCC. Disease duration ranged from 3 months to 8 years. In assessing the overall status (blood pressure, pulse rate, temperature), no indexes of clinical and blood biochemical parameters of statistically significant changes after treatment were found. Two patients needed the 4^{th} application. The long term results of the treatment were assessed 3 and 6 months after the first application of the preparation. There were also no registered cases of therapeutic course interruption due to the development of any apparent toxicity (pain syndrome, skin toxicity) and/or any other negative effects related to the preparation application. Disease relapses were not registered. More rapid restoration of the physiological coloring of skin integument was shown when the preparation was used with addition of dimethylsulfoxide.

The most illustrative examples are shown in the epicrises 1-3.

Epicrisis 1

Patient BIN, age 56, the diagnosis—BCC, the disease lasts for 1 year, stage T1N0M0. Complaints of a neoplasm on the left part of the cheek which first appeared in the summer of 2008 and began to progress in size. Self-treatment did not bring any results. The objective observation showed that the focus is located on the left cheek skin, next to the nose; and is asymmetric and localized. Swelling edges on the periphery, scarred-atrophic changes with appearance of telangiectasia in the centre, single albescent scales on the periphery of the focus were found visually. Surface smooth and lustrous. Focus painful, no itching. Treated with the preparation produced as in the ex. 4-1. The dark brown crust was formed in the place of treatment on the 8-th day, the rejection of which was completed on the 22-nd day. Pale pink spot in the place of treatment is observed, complete regeneration of epithelium is registered on the 80-th day of observation. Complete clinical and biochemical blood test were found without apparent changes, pulse rate, temperature and blood pressure were normal. During next 3 and 6 months the follow-up observation didn't show any unwanted sequelae or abnormal changes. The treatment is completed.

Epicrisis 2

Patient KEB, age 50, the diagnosis—ulcerous dermatitis covered with crust, the disease lasts for 2 years. Process of proliferative type, limited, localized on the skin of the left wing of the nose, introduced by pink colored nodules with ulcerations in the center, round shape, size 1*1 mm, boundaries are clear cut and indurated. The periphery is pearl-edged. Three applications of the preparation obtained as described in 2-10 have been performed. The crust persists up to the 22-nd day of observation, further rejection is without complications and is completed on the 3-d month. The examination of the treated area 6 months after the first application of the preparation showed no signs of the disease. The treatment is completed.

Epicrisis 3

Patient KUN, age 72, the diagnosis—multiple basalioma, relapsed. Considers to be ill since 2007, when a nodule on the tip of the nose was first noticed. The constant growth and appearance of the second nodule are observed since the beginning of 2008. In August 2008 underwent the photoradiation therapy at Moscow Regional Research and Clinical Institute “MONIKI” with positive results, but in May 2009 the appearance of a nodule in the same place was observed. Examination confirmed the presence of a pathological process, localized on the tip of the nose in the place of the old scar, non-inflammatory and non-diffused. Nodule of hemispheric shape, rose-tinted, 6 mm in diameter, indurated, not painful. The cytological screening of skin scrapings was carried out. The diagnostic material in the adjacent regions is of low pathology, scales of pavement epithelium and single basaloid structures can be discerned on the background of erythematic masses, suspicious of basalioma. Basaliomae were found in the nose skin scrapings. The application of the preparation obtained as described in 4-2 have been performed. In 24 hours the appearance of the crust was observed, upon removal of which the yellow incrustation is deposited. On the day 8 the crust on the tip of the nose is brown-yellow; upon removal the pyogenetic septic accretion appears. Light hyperemia persisted in the area of treatment on the 22-nd day of observation. The 4^{th} application was conducted. Blood tests, blood pressure, pulse rate and temperature are normal. Regeneration of the epithelium completed on the 53 day, and a small scar remained in application area by the 6-th month of observation. The check examination 9 and 12 months after the first visit showed no relapse of the disease. The treatment is completed.

The second test group involved 100 patients (25 men and 75 women) aged 18 to 86 diagnosed with: multiple papilloma (88 patients), seborrheic keratosis (31 patients), angiomas (also 22 patients with senile angioma), papillomatous nevi (18 patients) and cutaneous horn (4 patients). Seborrheic keratosis is the most frequent neoplasm, especially among elderly people.

The duration of the patient diseases ranged from 1.5 months to 35 years. For the patients with polypapilloma, angioma and nevus one application of the preparation is enough in the majority of cases. A single therapeutic dose is 0.01-0.2 ml. The treatment cycle is 1 course (3 weeks). The change of the tissue color occurred within 2-3 minutes after application of the pharmaceutical product, and with an acute hyperkeratosis—within 5-7 minutes. During application of the pharmaceutical product all patients felt a burning sensation in application areas that ceased within 10-12 minutes and did not require any pain management. Objectively, among the majority of patients the rim of hyperemia and infiltration around the treated tumor were observed and terminated spontaneously in 24-48 hours. The check examination after the 2-nd and the 8-th day of treatment revealed the treated tissues turned dark brown; significant induration and reduction in size were noticed.

In case of seborrheic keratosis and cutaneous horn an additional application of lesion focuses was conducted on 2-3 day and 7-9 day respectively.

After the application of the pharmaceutical product crust rejection usually occurred on the 5-th to the 19-th day; complete epithelialization finished in 1.5-2 months, the restoration of the physiological skin color—in 3-6 months. No pathological changes in tumors, or significant changes in the results of clinical and biochemical blood tests, or any relapse after conducted treatment were statistically observed.

The most typical examples are shown in epicrises 4-9.

Epicrisis 4

Patient BTA, born in 1974, complained of neoplasms on the skin of belly, back and axillary crease, which first appeared about 3 years ago. Has not undertaken any special treatment. Visual examination revealed numerous flesh-coloured elements; single ruby-colored hemispherical tumors were found on the abdomen skin on a thin base 0.1*0.1 cm in size. Complete blood count and biochemical blood assay results were within normal limits, blood pressure was normal. Diagnosis: papillomas, angiomas. After the application of the pharmaceutical product obtained as shown in the ex. 4-3, all elements became gray and indurated, a small erythema and infiltration appeared peripherally. The patient felt a slight burning sensation for 4-5 minutes. The next day all elements turned sorrel and partially mummified, peripheral erythema and infiltration remained On the 8^{th} day dark brown crusts were formed in the application area. Crust rejection was completed on the 13-th day. On the 22-d day only pink spots remained in application areas. Complete blood count and biochemical blood assay were without any visible changes, blood pressure was normal. Follow-up observation over the course of 3 and 6 months did not reveal any unwanted consequences and abnormal changes.

Epicrisis 5

Patient SNS, born in 1983, considers herself to be ill for about 2 years since occasional rash appeared in armpit area. In 2008 she had laser removal, but in 2 months there was a relapse accompanied by intensive growth of new elements. Visual examination revealed numerous flesh-colored thread-like elements on a thin base 0.2*0.2 cm in size, protruding above the skin surface. On the skin of her hands there were round tumors with acute keratosis The results of the complete blood count and biochemical blood assay were within normal limits, blood pressure was normal. Diagnosis: papillomas, warts. After application of the pharmaceutical product obtained as shown in the ex. p. 4-1, all the elements turned white and indurated immediately, a small erythema, oedema and infiltration appeared peripherally. The patient felt a slight burning sensation for about 10 minutes. The next day all the elements turned yellow-brown and partially mummified, peripheral erythema, oedema and infiltration partially remained. On the 5-th the dark-brown crust formation was observed in the treated areas, the rejection completed on the 9th day of treatment. By the 22-nd day only subtle pink spots could be seen in the treated area, the restoration of the physiological coloring of skin integument had been completed by the 3rd month of treatment. The complete blood count and biochemical blood assay were without any visible changes, blood pressure was normal. The follow-up observation over the course of 6 months didn't show any side effects or pathology. The treatment is completed.

Epicrisis 6

Patient TNG, born in 1941, considers herself to be ill for about 7 years, since occasional rash appeared in neck, hands and armpits areas. The disease is constantly progressing. In 2000, 2004, 2007 and 2008 she was treated by electrocoagulation method, but it was ineffective. Visual examination revealed numerous flesh-colored thread-like elements on a thin base 0.1*0.1 cm in size, and on a broad base with papillomatous surface; round tumors with acute keratosis are clearly observed on the hand skin. The results of the complete blood count and biochemical blood assay were within normal limits, blood pressure was normal. Diagnosis: papillomatous nevus, keratomas. After application of the pharmaceutical product obtained as shown in the ex. 4-4 all the elements turned gray and indurated. The patient experienced a light burning sensation for about 7 minutes. The next day all the elements considerably reduced in size, turned yellow-brown and partially mummified, slight peripheral erythema and infiltration were observed. On the 5-th day dark-brown crust formation and its partial rejection was observed in the treated area. No complaints. On the 22-nd day the complete epithelialization was observed in the treated area, the restoration of the physiological coloring of skin integument had been completed by the 3^{rd} month of treatment. The complete blood count and biochemical blood assay were without any visible changes, blood pressure was normal. The follow-up observation over the course of 6 months didn't reveal any side effects or pathology. The treatment is completed.

Epicrisis 7

Patient TMG, born in 1923, considers herself to be ill for 35 years, when solitary eruptions appeared on corpus and neck skin. The disease had been constantly progressing. In 1979 she had the treatment by the electrocoagulation method, in 2004, 2006 and 2009—by the cryodestruction method, but they didn't have any positive effect and new eruptions appeared. The visual examination showed numerous body-colour elements of thread-like shape with thin base 0.1*0.1 cm size and ruby-colour elements of hemisphere shape with clear boarders and also brown-colour elements with hyperkeratosis. Visual examination revealed numerous flesh-colored elements of a thread-like shape on the thin base 0.1*0.1 cm in size, ruby-colored elements of hemispherical shape with clear boundaries, as well as brown-colored elements with hyperkeratosis. The complete blood count and biochemical blood assay were within normal limits, blood pressure was normal. Diagnosis: papillomas, angiomas, keratomas. After the application of the product, obtained as shown in the ex. 4-5, all the elements turned white. The patient experienced light burning sensation for approximately 15 minutes. The following day all the elements indurated considerably, turned brown and partly mummified, peripherally a slight erythema and infiltration were observed. On the 8-th day the formation of dark brown crusts and their partial rejection was observed in the treated area. The patient had no complaints. On the 22-nd day complete epithelization was detected in the treated area. The complete blood count and biochemical blood assay showed no visible changes, blood pressure was normal. The follow-up observation over the course of 6 months revealed no adverse effects and no pathological changes.

Epicrisis 8

Patient BVI, born in 1933, considers herself to be ill for about 25 years, when solitary eruptions appeared on neck, armpits areas and corpus skin. The disease had been constantly progressing. In 1998 she was treated by the electrocoagulation method, in 2004 and 2006—by the cryodestruction method, in 2008—she had laser treatment, but they didn't have any positive effect and new lesions appeared. Visual examination of the neck and axillae revealed numerous flesh-colored elements of a thread-like shape, of the corpus skin—ruby-colored elements of hemispherical shape with clear boundaries and brown-colored elements with hyperkeratosis. The complete blood count and biochemical blood assay were within normal limits, blood pressure was normal. Diagnosis: papillomas, angiomas, seborrheic keratosis. After the application of the preparation, obtained as shown in the ex. 2-11, all the elements turned white and indurated. The patient experienced slight burning for approximately 15 minutes. The next day all the elements turned brown and partly mummified, peripherally a slight infiltration and erythema were observed. On the 8-th day the formation of dark brown crusts and their partial rejection were observed in the treated area. The patient had no complaints. On the 15-th day complete epithelization was observed in the treated area, and by the end of the 2^{nd} month, the restoration of the physiological coloring of skin integument was observed. The complete blood count and biochemical blood assay showed no visible changes, blood pressure was normal. The follow-up observation over the course of 6 months revealed no adverse effects and no pathological changes. The treatment is completed.

Epicrisis 9

Patient BNK, born in 1928, considers herself to be ill for about 10 years when solitary eruptions appeared on corpus and axillary cavity skin, and continue to appear and are constantly growing. In 2006 she was treated by the cryodestruction method, in 2009—by the laser method. The relapse is observed. Visual examination revealed numerous flesh-coloured papillomatous neoplasms in the axillae and on the side surfaces of the corpus, on the alvus's skin there are 3 ruby-coloured elements of hemispherical shape with evident hyperkeratinization 2*3 cm in size and numerous neoplasms with evident keratinization on the broad base. The complete blood count and biochemical blood assay were within normal limits, the blood tension was normal. Diagnosis: papillomas, a papillomatous nevus, angiomas, a cutaneous horn. After the application of the medicament, obtained as shown in the ex. 4-6 all the elements whitened and indurated. The patient experienced a light burning sensation for about 10 minutes. The following day all the elements turned brown and became partially mummified, a small erythema and oedema appeared peripherally. On the 8-th the dark brown crust formation and its partial rejection day in the treated area were observed. No complaints. On the 18-th day a complete epithelization was detected in the treated area, in the areas of crust rejection hyperpigmented spots were found. The complete blood count and biochemical blood assay showed no visible changes, the blood pressure was normal. The follow-up observation did not reveal any unwanted sequelae or abnormal changes. The treatment is completed.

Therefore, the clinical examination showed that the external application of the preparation arrests the proliferation of pathologically changed cells, provides for direct intravital fixation with the following mummification of pathologically changed tissue, doesn't leave any deep or interfacial cicatrices, is well tolerated by the patients, and is user friendly. The pharmaceutical product got State Marketing Authorization and is recommended for a wide usage in practical medicine for the removal of benign skin neoplasms: papillomatous nevuses, seborrheic keratosis, cutaneous horn, papillomatous benign skin disease caused by HPV, vascular benign skin disease and also in case of the relapses of these neoplasms after surgical, cryo—or laser treatment.

The invention is claimed as follows:

1. Pharmaceutical product obtained by interaction between selenium-containing compound and alpha, alpha-dichlorocarboxylic acid is distinguished by the fact that the selenious acid solution (with the amount of selenious acid no more than 20 pts. wt.) and alpha, alpha-dichloropropionic acid, stabilized by nitric acid (with the amount of nitric acid no more than 5 pts. wt.) are used in the interaction as selenium-containing compound.

2. Pharmaceutical product as in claim 1, is distinguished by the alpha, alpha-dichlorocarboxylic acid being the 2,2-dichloropropionic acid.

3. Pharmaceutical product as in claim 1, is distinguished by the amount of nitric acid being 1-3 pts. wt.

4. Pharmaceutical product as in claim 1, is distinguished by the amount of selenious acid being 0.5-10 pts. wt.

5. Pharmaceutical product as in claim 1 or 2, is distinguished by the fact that it contains additional 5-20% of dimethylsulfoxide.

6. Method of production of the pharmaceutical product as in claim 1 by interaction between selenium-containing compound and alpha, alpha-dichlorocarboxylic acid with temperature no higher than 70° C., is distinguished by the fact that the selenious acid solution (with the amount of selenious acid of 0.5-20 pts. wt.) and alpha, alpha-dichloropropionic acid, stabilized by nitric acid (with the amount of nitric acid no more than 5 pts. wt.) are used in the interaction as selenium-containing compound.

7. Method as in claim 4, is distinguished by the fact that the interaction is carried out at 20-30° C.

8. Method as in claim 4 or 5, is distinguished by the amount of nitric acid which is 1-3 pts. wt.

9. Method of application of the pharmaceutical product as in claim 1 for treatment of benign, viral, pre-malignant and malignant non-metastatic skin lesions, dysplastic lesions of visible mucous coats and other skin lesions, is distinguished by the fact that it is applied to the lesion focus on days 1, 2-3, 7-9 and 22-23 of treatment.
